(19) United States
(12) Patent Application Publication
(10) Pub. No.: US 2006/0189822 A1
(43) Pub. Date: Aug. 24, 2006

(54) Dispersant for dispersing carbon nanotubes and carbon nanotube composition comprising the same

(76) Inventors: Seon Mi Yoon, Yongin-Si (KR); Jae Young Choi, Suwon-Si (KR); Eun Sung Lee, Seoul (KR); Do Yun Kim, Seongnam-Si (KR); Yong Kyun Lee, Yongin-Si (KR)

Correspondence Address: CANTOR COLBURN, LLP, 55 GRIFFIN ROAD SOUTH, BLOOMFIELD, CT, 06002, US

(21) Appl. No.: 11/337,154

(22) Filed: Jan. 20, 2006

(30) Foreign Application Priority Data
Jan. 20, 2005 (KR) 2005-5443
Dec. 21, 2005 (KR) 2005-126825

(51) Int. Cl. C07C 69/76

(52) U.S. Cl. 560/130; 977/742

(57) ABSTRACT

Disclosed herein is a dispersant for dispersing carbon nanotubes which comprises a head having a high affinity for carbon nanotubes and a tail having a high affinity for a dispersion medium. The head includes an aromatic hydrocarbon group with a high affinity for carbon nanotubes to prevent aggregation of the carbon nanotubes, thereby improving the dispersibility of the carbon nanotubes. Further disclosed is a carbon nanotube composition comprising the dispersant.

### CROSS-REFERENCE TO RELATED PATENT APPLICATION

This non-provisional application claims priority under 35 U.S.C. §119(a) to Korean Patent Application No. 2005-5443 filed on Jan. 20, 2005 and Korean Patent Application No. 2005-126825 filed on Dec. 21, 2005, each of which is herein incorporated by reference in its entirety.

### BACKGROUND OF THE INVENTION

1. Field of the Invention

The present invention relates to a dispersant for dispersing carbon nanotubes and a carbon nanotube composition comprising the dispersant. More particularly, the present invention relates to a dispersant for dispersing carbon nanotubes which comprises a head including an aromatic hydrocarbon group with a high affinity for carbon nanotubes to prevent aggregation of the carbon nanotubes, thereby improving the dispersibility of the carbon nanotubes, and a carbon nanotube composition comprising the dispersant.

2. Description of the Related Art

Carbon nanotubes (CNTs) are tubes made of carbon atoms wherein one carbon atom is bonded to other adjacent carbon atoms in a hexagonal honeycomb pattern, and have an extremely small diameter in the nanometer range. Based on these structural characteristics, carbon nanotubes show unique electrochemical properties. In addition, carbon nanotubes have superior mechanical properties, better electrical selectivity, excellent field emission properties, high efficiency as hydrogen reservoirs, and the like. Furthermore, carbon nanotubes exhibit semiconductor properties according to their rolled shapes, have different energy gaps depending on their diameters, and exhibit specific quantum effects due to their quasi-one-dimensional structure. Thus, carbon nanotubes have drawn considerable attention in the fields of electronics, biotechnologies and medicines. For example, carbon nanotubes are used in the formation of conductive films and as materials for probes of field emission displays (FEDs) and scanning probe microscopes (SPMs). Under such circumstances, extensive research on carbon nanotubes is actively underway for a variety of applications.

Carbon nanotubes must be effectively dispersed in a matrix, such as a solution or a binder, in order to be used in the formation of conductive films and the fabrication of a variety of electronic devices. However, carbon nanotubes tend to aggregate together to form a bundle of the carbon nanotubes and collide with each other in a matrix due to a strong Van der Waals force. If carbon nanotubes aggregate together in a matrix, problems may arise that the inherent characteristics of the carbon nanotubes are not sufficiently exhibited and the uniformity of thin films formed of the carbon nanotubes are deteriorated.

Due to the inherent characteristics of carbon nanotubes, no commercially available dispersants have succeeded in sufficiently dispersing carbon nanotubes. Various attempts have been made to develop a dispersant for uniformly dispersing or dissolving carbon nanotubes in a solvent or a resin.

For example, U.S. Pat. No. 6,787,600 describes a dispersant which comprises a polyamine (e.g., polyallylamine) or polyimine (e.g., polyethyleneimine) backbone chain containing side chains of two or more different types of polyester chain. The dispersant has a structure wherein two or more different types of polyester side chain are bonded to a linear backbone chain.

U.S. Pat. No. 6,599,973 describes an aqueous graft copolymer for a pigment having a weight average molecular weight of 5,000 to 100,000 and comprising a hydrophobic polymeric backbone and anionic and nonionic hydrophilic side chains attached to the backbone.

U.S. Pat. No. 5,530,070 describes an aqueous metallic flake dispersant wherein the dispersant is formed by polymerizing ethylenically unsaturated monomers and has macromonomer side chains attached to a polymeric backbone.

However, these references fail to teach satisfactory dispersion of carbon nanotubes. Further, since most of the conventional dispersants are polymeric dispersants, they have the disadvantages of low solubility and high viscosity, making it impossible to sufficiently disperse carbon nanotubes. There is thus a need to develop a new dispersant for dispersing carbon nanotubes that is capable of preventing the carbon nanotubes from colliding and aggregating together.

### SUMMARY OF THE INVENTION

Therefore, the present invention has been made in view of the above problems of the previously available dispersants, and it is one object of embodiments of the present invention to provide a dispersant for dispersing carbon nanotubes that is capable of preventing the carbon nanotubes from aggregating and thus the dispersibility of carbon nanotubes is improved.

It is another object of embodiments of the present invention to provide a carbon nanotube composition in which the dispersibility of carbon nanotubes is improved so that the characteristics of the carbon nanotubes are sufficiently exhibited.

In accordance with one aspect of embodiments of the present invention for achieving the above objects, there is provided a dispersant for dispersing carbon nanotubes comprising a head and a tail linked to the head wherein the tail includes at least one repeating unit of Formula 1 below and at least one repeating unit of Formula 2 below, or is polyethylene oxide or polypropylene oxide; and the head is a substituted or unsubstituted C₆₋₃₀ aromatic hydrocarbon group:

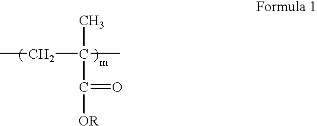

wherein m is from 3 to 30, and R is selected from the group consisting of C₁₋₁₀ alkyl, alkenyl and alkynyl groups,

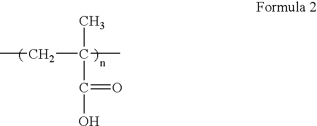

wherein n is from 3 to 30.

The dispersant for dispersing carbon nanotubes according to embodiments of the present invention may have a structure of Formula 3 or 4 below:

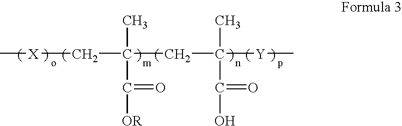

wherein X and Y are identical to or different from each other and are each independently a C₆₋₃₀ homocyclic or heterocyclic aromatic hydrocarbon group which is unsubstituted or substituted with at least one halogen atom,

R is selected from the group consisting of C₁₋₁₀ alkyl, alkenyl and alkynyl groups,

m and n are each independently from 3 to 20, the ratio m/n being from 3 to 12, and

o and p are each independently 0 or 1, with the proviso that both o and p are not zero;

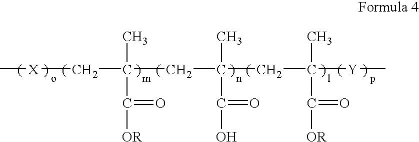

wherein X and Y are identical to or different from each other and are each independently a C₆₋₃₀ homocyclic or heterocyclic aromatic hydrocarbon group which is unsubstituted or substituted with at least one halogen atom,

R is selected from the group consisting of C₁₋₁₀ alkyl, alkenyl and alkynyl groups,

m, n and l are each independently from 3 to 20, the ratio m+l/n being from 3 to 12, and

o and p are each independently 0 or 1, with the proviso that both o and p are not zero.

In accordance with another aspect of embodiments of the present invention, there is provided a carbon nanotube composition comprising the dispersant, carbon nanotubes, and a solvent.

### BRIEF DESCRIPTION OF THE DRAWINGS

The above and other objects, features and other advantages of embodiments of the present invention will be more clearly understood from the following detailed description taken in conjunction with the accompanying drawings, in which:

FIG. 1 is a diagram schematically showing a state wherein carbon nanotubes are dispersed in a dispersion medium using a dispersant for dispersing carbon nanotubes according to embodiments of the present invention;

FIG. 2 is a graph showing the absorbance of carbon nanotube solutions prepared in Examples 1-4 according to the kind of dispersants used in the carbon nanotube solutions;

FIG. 3 is a graph showing the absorbance of carbon nanotube solutions prepared in Examples 5 and 6 and Comparative Examples 1 and 2 according to the kind of dispersants used in the carbon nanotube solutions; and

FIG. 4 is a graph comparing the absorbance of carbon nanotube solutions prepared in Examples 7-15 and Comparative Example 3.

### DETAILED DESCRIPTION OF THE PREFERRED EMBODIMENTS

Hereinafter, embodiments of the present invention will be explained in more detail with reference to the accompanying drawings.

The head of the dispersant according to embodiments of the present invention includes an aromatic hydrocarbon group having an affinity for carbon atoms so as to allow the head to have an affinity for carbon nanotubes as dispersoids.

The dispersant for dispersing carbon nanotubes according to embodiments of the present invention comprises a head including an aromatic hydrocarbon group having a high affinity for carbon nanotubes and a tail having a high affinity for a dispersion medium. Since the dispersant of embodiments of the present invention is a low-molecular weight organic substance, it is decomposed at a temperature lower than the firing temperatures of general films. Accordingly, when a carbon nanotube composition comprising the dispersant of embodiments of the present invention is formed into a thin film, no organic material substantially remains after firing and thus the thin film has uniform physical properties without being contaminated. In addition, the dispersant of embodiments of the present invention has a higher solubility and a lower viscosity than conventional polymeric dispersants. Since the dispersant of embodiments of the present invention is composed of the same elements as those of carbon nanotubes, there is no or very little contamination by other elements.

The aromatic hydrocarbon group constituting the head is structurally similar to carbon nanotubes, and can form π-π bonds between carbon nanotubes and π electrons of the head of the dispersant. For these reasons, the head can have an affinity for carbon nanotubes. Accordingly, the use of the dispersant comprising the head facilitates dispersion of substantially indispersible carbon nanotubes in any dispersion medium.

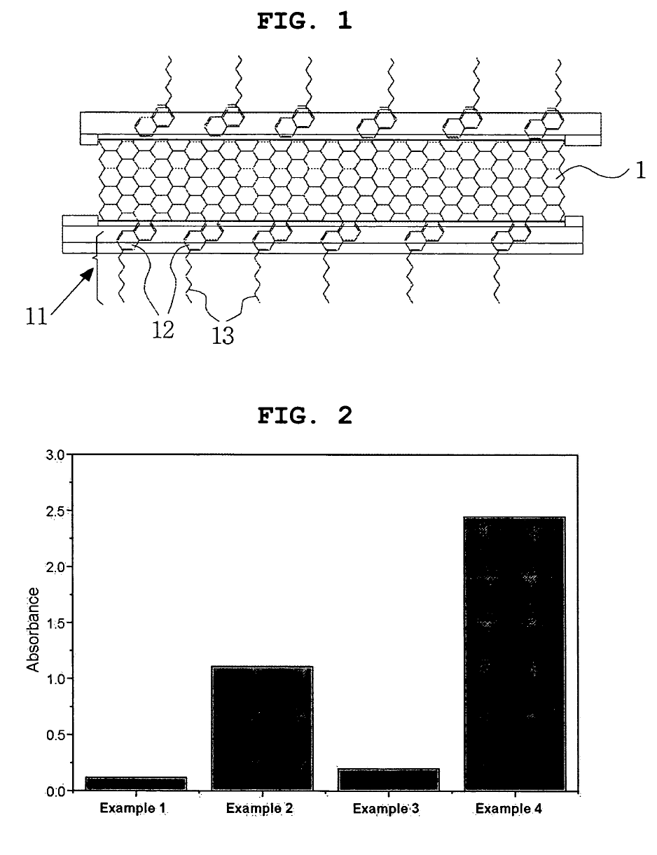

FIG. 1 is a diagram schematically illustrating the operational mechanism of the dispersant according to embodiments of the present invention for dispersing carbon nanotubes. Specifically, the figure shows a state wherein carbon nanotubes are dispersed in a dispersion medium using the dispersant of embodiments of the present invention. Referring to FIG. 1, each dispersant 11 for dispersing carbon nanotubes according to embodiments of the present invention comprises a head 12 having a high affinity for carbon nanotubes 1 and a tail 13 having a high affinity for a dispersion medium. It is assumed that the head 12 of the dispersant 11 is adsorbed to the carbon nanotubes 1 by π-π interaction with π electrons of the carbon nanotubes 1, thereby solubilizing the carbon nanotubes 1. At this time, the heads 12 of the dispersants 11 are aligned along the carbon nanotubes 1. The tails 13 having an affinity for a dispersion medium and centered on the heads 12 are scattered in the dispersion medium, enabling dispersion of the carbon nanotubes in the dispersion medium.

The aromatic hydrocarbon group constituting the head of the dispersant according to embodiments of the present invention is C₆₋₃₀ aromatic hydrocarbon group. The aromatic hydrocarbon group is at least one benzene ring unsubstituted or substituted with at least one halogen atom.

Aromatic rings of the aromatic hydrocarbon group have various conformations and interact with each other. For example, unsubstituted aromatic rings have an edge-face interaction, and substituted aromatic rings have an offset stacked or face-face stacked geometry interaction. The direction of aromatic rings greatly affects the intensity of interaction between the aromatic rings. In the case where the aromatic hydrocarbon group is substituted with at least one halogen atom, e.g., fluorine atom, face-face stacked geometry interaction is enhanced, leading to a further improvement in dispersibility.

Examples of preferred aromatic hydrocarbon groups that can constitute the head of the dispersant according to embodiments of the present invention include, but are not limited to, benzene, and polycyclic aromatic hydrocarbon groups, including naphthalene, imidazole, acenaphthalene, acenaphthene, fluorene, anthracene, phenanthrene, fluoranthene, pyrene, benzanthracene, chrysene, benzo(b)fluoranthene, benzo(k)fluoranthene, benzopyrene, benzoperylene, indeno(1,2,3-c,d)pyrene, etc.

Tails that can be linked to the head of the dispersant according to embodiments of the present invention may be properly selected among those having an affinity for dispersion medium to be used.

The tail may be a polyacrylic-based repeating unit including at least one hydrophobic repeating unit of Formula 1 below and at least one hydrophilic repeating unit of Formula 2 below, or is polyethylene oxide or polypropylene oxide:

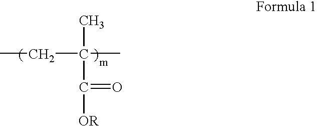

wherein m is from 3 to 30, and R is selected from the group consisting of C₁₋₁₀ alkyl, alkenyl and alkynyl groups:

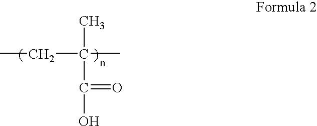

wherein n is from 3 to 30.

In the case where the tail is a polyacrylic-based repeating unit, the hydrophilic repeating unit and the hydrophobic repeating unit are sequentially linked to the head, or vice versa. Alternatively, the hydrophilic repeating unit and the hydrophobic repeating unit may be linked to the head in an alternating manner. Alternatively, one hydrophilic repeating unit is linked to the head and then two hydrophobic repeating units may be linked thereto. The range of solvents usable in embodiments of the present invention can be broadened by adjusting the ratio between the number of the hydrophilic repeating unit and that of the hydrophobic repeating unit.

The tail can be appropriately selected depending on the characteristics of dispersion medium to be used. Preferred tails are selected from the group consisting of, but are not limited to, polymethylmethacrylate, polybutylmethacrylate, polyacrylic acid, polymethacrylic acid, polyoxyethylene, polyoxypropylene, and copolymers of polyalkylmethacrylate and polymethacrylic acid.

Specifically, one example of the dispersant for dispersing carbon nanotubes according to embodiments of the present invention has a structure of Formula 3 or 4 below:

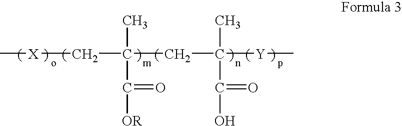

wherein X and Y are identical to or different from each other and are each independently a C₆₋₃₀ homocyclic or heterocyclic aromatic hydrocarbon group which is unsubstituted or substituted with at least one halogen atom,

R is selected from the group consisting of C₁₋₁₀ alkyl, alkenyl and alkynyl groups,

m and n are each independently from 3 to 20, the ratio m/n being from 3 to 12, and

o and p are each independently 0 or 1, with the proviso that both o and p are not zero;

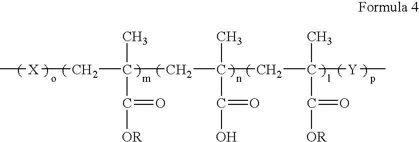

wherein X and Y are identical to or different from each other and are each independently a C₆₋₃₀ homocyclic or heterocyclic aromatic hydrocarbon group which is unsubstituted or substituted with at least one halogen atom,

R is selected from the group consisting of C₁₋₁₀ alkyl, alkenyl and alkynyl groups,

m, n and l are each independently from 3 to 20, the ratio m+l/n being from 3 to 12, and

o and p are each independently 0 or 1, with the proviso that both o and p are not zero.

Preferred dispersants for dispersing carbon nanotubes according to embodiments of the present invention include, but are not limited to, those represented by Formulae 5 to 8 below:

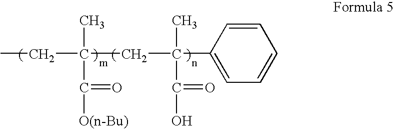

wherein m and n are each independently from 3 to 20, the ratio m/n being 3-12;

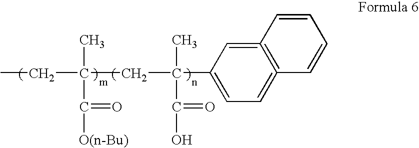

wherein m and n are each independently from 3 to 20, the ratio m/n being 3-12;

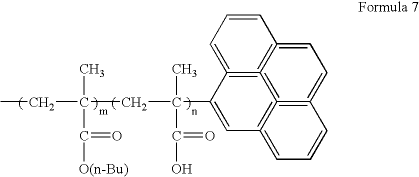

wherein m and n are each independently from 3 to 20, the ratio m/n being 3-12;

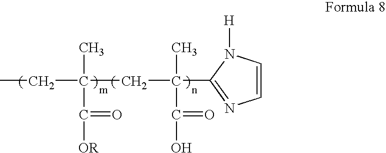

wherein m and n are each independently from 3 to 20, the ratio m/n being 3-12.

The dispersant whose tail is polyethylene oxide or polypropylene oxide may have a structure represented by Formula 9 below:

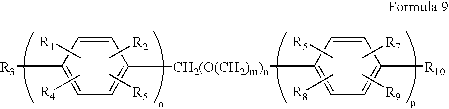

wherein R₁ to R₁₀ are identical to or different from each other and are each independently a hydrogen or fluorine atom,

m is 2 or 3,

n is from 5 to 22,

o and p are each independently 0 or 1, with the proviso that both o and p are not zero.

Examples of preferred dispersants of Formula 9 include structures represented by Formulae 10 to 14 below:

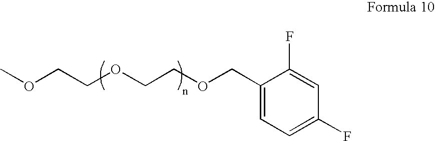

wherein n is from 3 to 20;

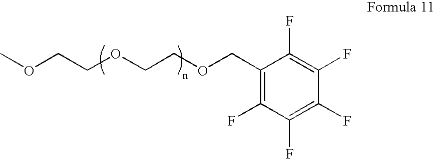

wherein n is from 3 to 20;

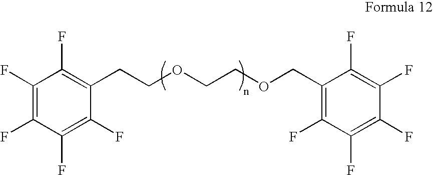

wherein n is from 3 to 20;

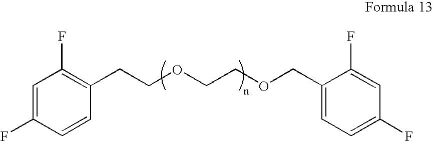

wherein n is from 3 to 20;

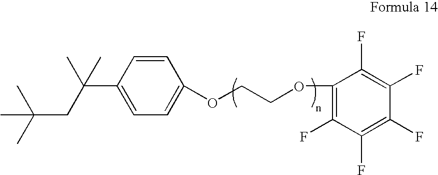

wherein n is from 3 to 20.

In the case where the aromatic rings constituting the head are substituted with fluorine atoms, face-face stacked interaction between the aromatic rings is enhanced, leading to a further improvement in dispersibility.

The dispersant of embodiments of the present invention may have a structure wherein aromatic hydrocarbon groups are linked to both ends of the tail. In this case, since at least one head of the two heads can be linked to the tail in an equilibrium reaction, the dispersion stability of the dispersant can be improved. Specific examples of preferred dispersants having such structures include compounds of Formulae 12 and 13 below:

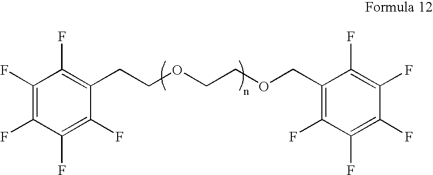

wherein n is from 3 to 20;

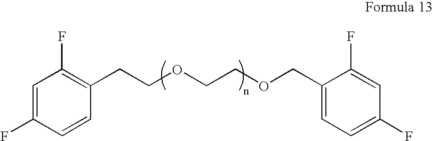

wherein n is from 3 to 20.

The dispersant having aromatic hydrocarbon groups linked to both ends of the tail may have an asymmetric structure wherein one of the aromatic hydrocarbon groups linked to one end of the tail is unsubstituted and the other aromatic hydrocarbon group linked to the other end of the tail is substituted with at least one substituent, e.g., fluorine atom. The aromatic rings may be linked with at least one straight or branched alkyl group. One example of such dispersants may have a structure represented by Formula 14 below:

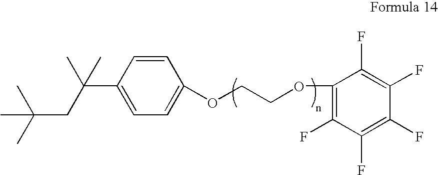

wherein n is from 3 to 20.

Dispersants of embodiments of the present invention can be synthesized as follows. The tail of the dispersants of Formulae 5 to 8 can be prepared by using n-butyl methacrylate (n-BMA) and trimethylsilyl methacrylate (TMSMA) as monomer raw materials according to Reaction Scheme 1. In this case, n-butyl is used as a side chain. When it is intended to change the kind of side chains, raw materials containing the corresponding side chains can be used as starting materials.

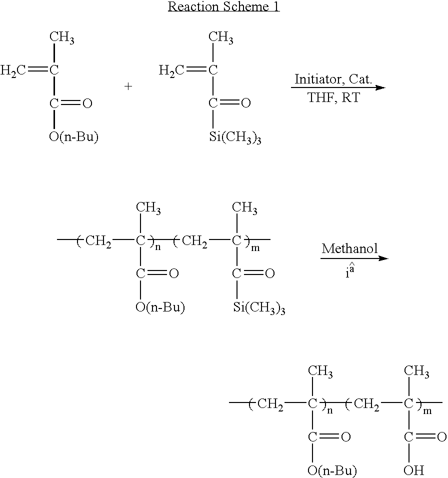

The tail thus prepared reacts with a raw material for a head to prepare a dispersant in which the tail is bonded to the head. Thereafter, methanol is added to the solution, and refluxed for 6 hours. The solvents are removed under reduced pressure to substitute the silyl group of the trimethylsilyl methacrylate with a carboxyl group, completing the synthesis of the desired dispersant.

A dispersant according to another embodiment of embodiments of the present invention wherein the head is a substituted aromatic hydrocarbon group can be synthesized according to Reaction Scheme 2. As depicted in Reaction Scheme 2, Compound 1 constituting a tail is added dropwise to a solution of NaH in THF under an Ar atmosphere at room temperature. Subsequently, Compound 2 is added dropwise to the mixture in ice-water, and then the resulting reaction mixture is stirred at room temperature. The reaction mixture is extracted with H₂O and methylene chloride, and the extracted mixture is purified through a column, yielding the desired dispersant in which the head is a fluorine-substituted aromatic hydrocarbon.

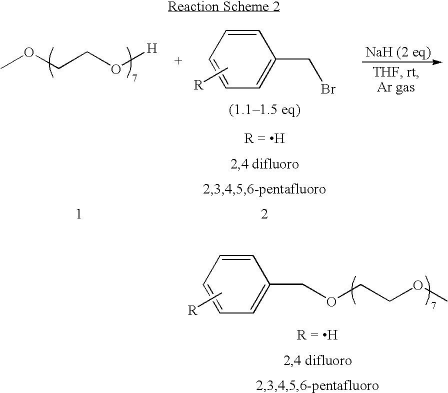

Embodiments of the present invention are directed to carbon nanotube compositions comprising the dispersant. The carbon nanotube compositions comprise carbon nanotubes, the dispersant, and a solvent.

Examples of suitable carbon nanotubes that can be used in the composition of embodiments of the present invention include, but are not limited to, single-walled carbon nanotubes, double-walled carbon nanotubes, multi-walled carbon nanotubes, rope carbon nanotubes, and combinations thereof.

Examples of preferred solvents that can be used in the composition of embodiments of the present invention include, but are not limited to: water; alcohols, e.g., methanol, ethanol, isopropyl alcohol, propyl alcohol and butanol; ketones, e.g., acetone, methyl ethyl ketone, ethyl isobutyl ketone and methyl isobutyl ketone; glycols, e.g., ethylene glycol, ethylene glycol methyl ether, ethylene glycol mono-n-propyl ether, propylene glycol, propylene glycol methyl ether, propylene glycol ethyl ether, propylene glycol butyl ether and propylene glycol propyl ether; amides, e.g., dimethylformamide and dimethylacetamide; pyrrolidones, e.g., N-methylpyrrolidone and N-ethylpyrrolidone; dimethylsulfoxide; γ-butyrolactone; hydroxyesters, e.g., methyl lactate, ethyl lactate, methyl β-methoxyisobutyrate and methyl α-hydroxyisobutyrate; anilines, e.g., aniline and N-methylaniline; hexane; terpineol; chloroform; toluene; propylene glycol monomethyl ether acetate (PGMEA); and N-methyl-2-pyrrolidone (NMP).

The carbon nanotube composition of embodiments of the present invention comprises 0.001 to 10 parts by weight of the dispersant, 0.01 to 5 parts by weight of the carbon nanotubes and 85 to 99.989 parts by weight of the solvent (i.e. dispersion medium), based on 100 parts by weight of the composition. It is preferred that the mixing weight ratio of the carbon nanotubes to the dispersant be between 1:0.001 and 1:10.

The carbon nanotube composition of embodiments of the present invention may further comprise a binder or an organic additive.

The carbon nanotube composition of embodiments of the present invention can be prepared by mixing the carbon nanotubes, the dispersant and the solvent using a stirring or mixing apparatus, e.g., a sonic bath, a homogenizer, a spiral mixer, a spindle mixer, a disperser, or a hybrid mixer.

According to the carbon nanotube composition of embodiments of the present invention, the carbon nanotubes can be sufficiently dispersed in a matrix, such as a solution or resin, without deteriorating the characteristics of the carbon nanotubes. In addition, the carbon nanotube composition of embodiments of the present invention exhibits superior dispersion stability without separation or aggregation of the carbon nanotubes even after long-term storage. Furthermore, the carbon nanotube composition of embodiments of the present invention is superior in terms of conductivity, film-formation properties and moldability.

The carbon nanotube composition of embodiments of the present invention can be applied to a substrate by simple coating techniques, including spin coating, electrophoresis, casting, ink-jet printing, spraying, and offset printing. The carbon nanotube composition of embodiments of the present invention can be used as a material for an electron gun or an electrode of a field emission display (FED), a transparent electrode of an electroluminescence display or a liquid crystal display, or a light-emitting material, a buffer material, an electron transport material or a hole transport material of an organic electroluminescence device, or the like.

Embodiments of the present invention will now be described in more detail with reference to the following examples. However, the examples are given for the purpose of illustration and are not to be construed as limiting the scope of the invention.

### EXAMPLES

### Preparative Example 1

i) Preparation of Tail

To prepare a block copolymer backbone chain having n-butylmethacrylate (BMA) and trimethylsilylmethacrylate (TMSA) (composition ratio=5:1) as repeating units and an estimated molecular weight of 1,000, dimethylketene methyltrimethylsilyl acetal (3.48 g, 20 mmol) as an initiator and tetrabutylammonium-chlorobenzoate (0.8 g, 0.2 mmol) as a catalyst were dissolved in acetonitrile (0.5 ml), and stirred using a magnetic stirring bar for 2 hours in a round-bottom flask. To the solution were slowly added n-butylacrylate (8.18 g, 57.5 mmol) as a first monomer and tetrahydrofuran (THF) (2 ml). After the reaction was continued for 4 hours, analysis of gas chromatography showed that the first monomer disappeared. Thereafter, a mixture of trimethylsilylmethacrylate (1.82 g, 11.5 mmol) as a second monomer and tetrahydrofuran (1 ml) was slowly added. After the reaction mixture was allowed to react for 2 hours, analysis of gas chromatography showed that the second monomer disappeared.

ii) Introduction of Head

A solution of tetrabutylammonium-chlorobenzoate (0.8 g, 0.2 mmol) as a catalyst in acetonitrile (0.5 ml) was added to the tail, followed by the addition of benzaldehyde (1.59 g, 15 mmol). The mixture was allowed to react for 6 hours to prepare a solution.

iii) Substitution with Carboxyl Group

To substitute the silyl group of the trimethylsilylmethacrylate with a carboxyl group, methanol is added to the solution, refluxed for 6 hours, and evaporated under reduced pressure to remove the solvents. The residue was dried in a vacuum oven for 24 hours, yielding a dispersant as a viscous oil.

### Preparative Examples 2-19

Dispersants for dispersing carbon nanotubes having various structures were prepared by varying the kind of tails and heads, the ratio of raw materials for the tails and the addition order as indicated in Table 1 below.

TABLE 1

| Preparative | | |
|---|---|---|
| Example | Tail | Head |
| 2 | BMA/TMSMA = 1/1 | Benzaldehyde |
| 3 | BMA/TMSMA = 3/1 | Benzaldehyde |
| 4 | BMA/TMSMA = 5/1 | Benzaldehyde |
| 5 | BMA/TMSMA = 7/1 | Benzaldehyde |
| 6 | BMA/TMSMA = 5/1 | 2-Naphthaaldehyde |
| 7 | BMA/TMSMA = 5/1 | 1-Pyrenecarboxyaldehyde |
| 8 | BMA/TMSMA = 5/1 | 2-Imidazolecarboxyaldehyde |
| 9 | BMA/TMSMA = 5/1 | Benzaldehyde |
| 10 | TMSMA/BMA = 1/5 | Benzaldehyde |
| 11 | BMA/TMSMA/ | Benzaldehyde |
| | BMA = 2.5//1//2.5 | |
| 12 | TMSMA/BMA// | 4-Acetoxybenzaldehyde |
| | BMA = 3/0.67//1.33 | |
| 13 | BMA/TMSMA = 5/1 | 4-(Methylthio)benzaldehyde |
| 14 | BMA/TMSMA = 5/1 | 2-(Methylthio)ethylmethacrylate |
| 15 | BMA/TMSMA = 1/1 | 4-AcetoxyBenzaldehyde |
| 16 | BMA/TMSMA = 1/1 | 2-Naphthaaldehyde |
| 17 | BMA/TMSMA = 1/1 | 1-Pyrenecarboxyaldehyde |
| 18 | BMA/TMSMA = 1/1 | 2-Imidazolecarboxyaldehyde |
| 19 | TMSMA/BMA = 1/0.5 | Benzylmethacrylate |

* BMA: n-Butylmethacrylate
** TMSMA: Trimethylsilylmethacrylate

### Preparative Example 22

### Preparation of Dispersant

As depicted in Reaction Scheme 1, Compound 1 (1 eq., 0.34 g) was added dropwise to a solution of NaH (1.2 eq., 0.0288 g) in THF (3 ml/mmol) under an Ar atmosphere at room temperature. Subsequently, Compound 2 (1.06 eq., 0.2046 g) was added dropwise to the mixture in ice-water, and stirred at room temperature for 24 hours. The reaction mixture was extracted with H₂O (20 ml/mmol) and methylene chloride (20 ml×3). The extracted mixture was purified by silica gel column chromatography (MC/n-Hexane (7/3) followed by MC/MeOH (9/1)), giving the dispersant of Formula 10 (yield: 65-83%).

### Examples 1-4

200 mg of each of the dispersants represented by Formula 15 (Example 1), Formula 16 (Example 2), Formula 17 (Example 3) and Formula 18 (Example 4) was dissolved in 20 g of NMP. 20 mg of single-walled carbon nanotubes was added to the solution, dispersed in a sonic bath (35 kHz, 400 W) for 13 hours, centrifuged at 5,000 rpm for 10 minutes, followed by at 8,000 rpm for 10 minutes to prepare a carbon nanotube solution. Aggregated powders were removed from the carbon nanotube solution. The absorbance of the solution was measured at 700 nm by UV-Vis-spectroscopy (JASCO (V-560), Absorbance mode, Scanning speed: 400 nm/min.). The results are shown in FIG. 2. The graph shown in FIG. 2 reveals that the dispersants have a high absorbance, which indicates that the carbon nanotubes were highly dispersed.

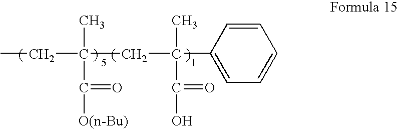

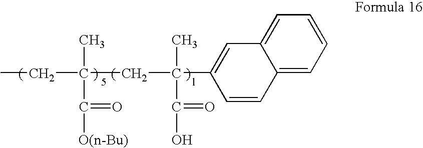

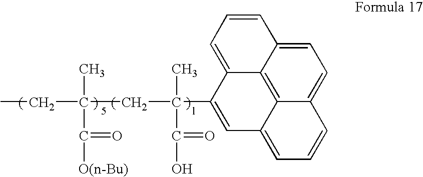

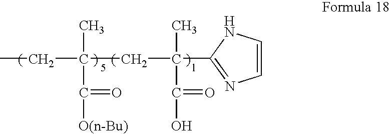

### Examples 5 and 6

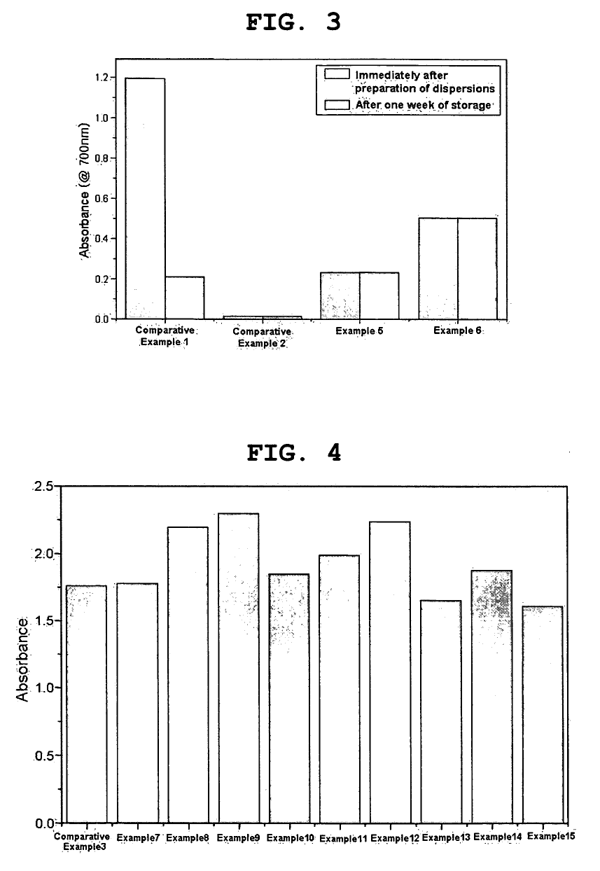

200 mg of each of the dispersants represented by Formula 16 (Example 5) and Formula 18 (Example 6) was dissolved in 20 g of terpineol. 20 mg of single-walled carbon nanotubes was added to the solution, dispersed in a sonic bath (35 kHz, 400 W) for 13 hours, and centrifuged at 8,000 rpm for 10 minutes to prepare a carbon nanotube solution. Aggregated powders were removed from the carbon nanotube solution. The absorbance of the solution was measured at 700 nm by UV-Vis-spectroscopy (JASCO (V-560), Absorbance mode, Scanning speed: 400 nm/min.). The results are shown in FIG. 3.

### Comparative Examples 1 and 2

Carbon nanotube solutions were prepared in the same manner as in Example 5, except that the dispersants of Formula 19 (Comparative Example 1) and Formula 20 (Comparative Example 2) were used. The absorbance of the carbon nanotube solutions was measured at 700 nm by the same method as in Example 5. The obtained results are shown in FIG. 3.

The graph shown in FIG. 3 demonstrates that the dispersants of embodiments of the present invention have a high absorbance, indicating that the carbon nanotubes were dispersed in high concentration and the dispersed states were stably maintained with the passage of time. In contrast, the dispersant of Comparative Example 1 showed superior dispersibility immediately after preparation of the dispersion, but the absorbance of the dispersion was drastically decreased after one week of storage. The dispersant of Comparative Example 2 had poor dispersibility, but exhibited some dispersion effects due to high affinity of the head for carbon nanotubes. If a solvent or binder system is changed, a dispersant having the tail of the dispersant of Formula 19 or 20 will be effective.

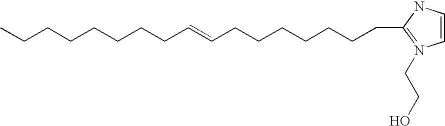

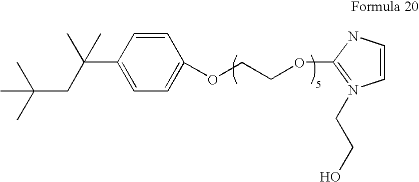

### Examples 7-15

20 mg of each of the dispersants whose tails were polyethylene glycol and whose heads were compounds shown in Table 2 was dissolved in 20 g of NMP. 2 mg of acid-treated single-walled carbon nanotubes was added to the solution, dispersed in a sonic bath for 12 hours, and centrifuged at 7,000 rpm for 10 minutes to prepare a carbon nanotube solution. The absorbance of the carbon nanotube solution was measured by the same method as in Example 1, and the obtained results are shown in FIG. 4.

TABLE 2

| Example No. | Head | Tail |
|---|---|---|
| Example 7 | Benzene | Polyoxyethylene |
| Example 8 | Naphthalene | |
| Example 9 | PentafluoroBenzene | |
| Example 10 | Bis(benzene) | |
| Example 11 | Bis(difluorobenzene) | |
| Example 12 | Fluorobenzene | |
| Example 13 | Pyrene | |
| Example 14 | Bis(pentafluorobenzene) | |
| Example 15 | Pentafluorobenzene | Octyl phenyl PEG |

| | |
|---|---|
| Comparative | Dispersant not used |
| Example 3 | |

### Comparative Example 3

2 mg of acid-treated single-walled carbon nanotubes was added to 20 g of NMP without the use of a dispersant, dispersed in a sonic bath (35 kHz, 400 W) for 12 hours, and centrifuged at 7,000 rpm for 10 minutes to prepare a carbon nanotube solution. The absorbance of the carbon nanotube solution was measured by the same method as in Example 1, and the obtained results are shown in FIG. 4.

The graph shown in FIG. 4 reveals that the carbon nanotubes were well dispersed by the dispersants of the present invention. Particularly, the dispersants in which the aromatic hydrocarbon groups constituting the heads were substituted with fluorine atoms improved the dispersibility of the carbon nanotubes.

Although the preferred embodiments of the present invention have been disclosed for illustrative purposes, those skilled in the art will appreciate that various modifications and variations are possible, without departing from the technical spirit of the invention. Accordingly, such modifications and variations are intended to come within the scope of the appended claims.

As apparent from the foregoing, the dispersant for dispersing carbon nanotubes according to embodiments of the present invention can prevent collision and aggregation of carbon nanotubes in a dispersion medium, thereby sufficiently dispersing the carbon nanotubes in the dispersion medium.

According to the carbon nanotube composition comprising the dispersant of embodiments of the present invention, the dispersibility of carbon nanotubes is improved so that the inherent electrochemical properties of the carbon nanotubes are sufficiently exhibited and uniform physical properties can be provided to thin films formed of the carbon nanotube composition.

Since the dispersant of embodiments of the present invention is decomposed at low temperature, no organic material substantially remains despite firing during formation of thin films using the carbon nanotube composition and thus deterioration in the characteristics of the thin films arising from contamination can be prevented.

What is claimed is:

1. A dispersant for dispersing carbon nanotubes comprising a head and a tail linked to the head
wherein the tail comprises at least one repeating unit of Formula 1 below or at least one repeating unit of Formula 2 below, or is polyethylene oxide or polypropylene oxide; and the head comprises a substituted or unsubstituted C₆₋₃₀ aromatic hydrocarbon group: 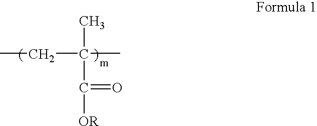
wherein m is from 3 to 30, and R is selected from the group consisting of C₁₋₁₀ alkyl, alkenyl and alkynyl groups, 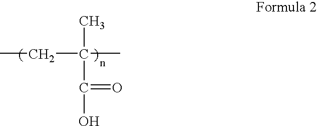
wherein n is from 3 to 30.

2. The dispersant according to claim 1, wherein the head is at least one group selected from the group consisting of benzene, and polycyclic aromatic hydrocarbon groups, including naphthalene, imidazole, acenaphthalene, acenaphthene, fluorene, anthracene, phenanthrene, fluoranthene, pyrene, benzanthracene, chrysene, benzo(b)fluoranthene, benzo(k)fluoranthene, benzopyrene, benzoperylene, and indeno(1,2,3-c,d)pyrene.

3. The dispersant according to claim 1, wherein the tail is at least one polymer selected from the group consisting of polymethylmethacrylate, polybutylmethacrylate, polyacrylic acid, polymethacrylic acid, polyoxyethylene, polyoxypropylene, and copolymers of polyalkylmethacrylate and polymethacrylic acid.

4. The dispersant according to claim 1, wherein the dispersant has a structure of Formula 3 or 4 below: 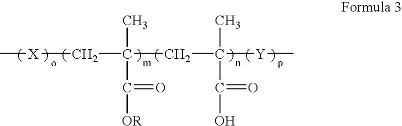
wherein X and Y are identical to or different from each other and are each independently a C₆₋₃₀ homocyclic or heterocyclic aromatic hydrocarbon group which is unsubstituted or substituted with at least one halogen atom,
R is selected from the group consisting of C₁₋₁₀ alkyl, alkenyl and alkynyl groups,
m and n are each independently from 3 to 20, the ratio m/n being from 3 to 12, and
o and p are each independently 0 or 1, with the proviso that both o and p are not zero; 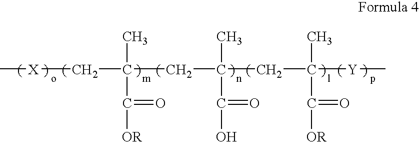
wherein X and Y are identical to or different from each other and are each independently a C₆₋₃₀ homocyclic or heterocyclic aromatic hydrocarbon group which is unsubstituted or substituted with at least one halogen atom,
R is selected from the group consisting of C₁₋₁₀ alkyl, alkenyl and alkynyl groups,
m, n and l are each independently from 3 to 20, the ratio m+l/n being from 3 to 12, and
o and p are each independently 0 or 1, with the proviso that both o and p are not zero.

5. The dispersant according to claim 4, wherein the dispersant has a structure represented by one of the following Formulae 5 to 8 below: 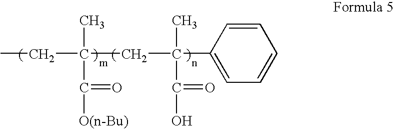
wherein m and n are each independently from 3 to 20, the ratio m/n being 3-12; 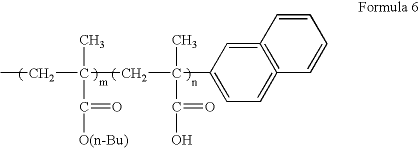
wherein m and n are each independently from 3 to 20, the ratio m/n being 3-12; 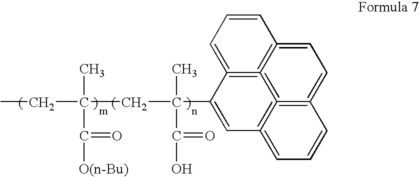
wherein m and n are each independently from 3 to 20, the ratio m/n being 3-12; 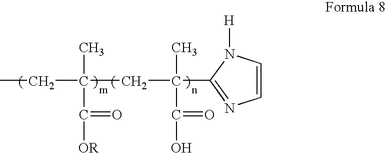
wherein m and n are each independently from 3 to 20, the ratio m/n being 3-12.

6. The dispersant according to claim 1, wherein the dispersant has a structure of Formula 9 below: 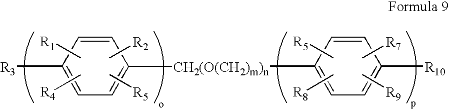
wherein R₁ to R₁₀ are identical to or different from each other and are each independently a hydrogen or fluorine-atom,
m is 2 or 3,
n is from 5 to 22,
o and p are each independently 0 or 1, with the proviso that both o and p are not zero.

7. The dispersant according to claim 6, wherein the dispersant has a structure represented by one of the following Formulae 10 to 14 below: 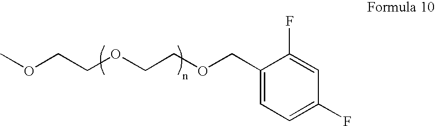
wherein n is from 3 to 20; 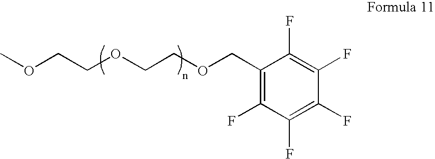
wherein n is from 3 to 20; 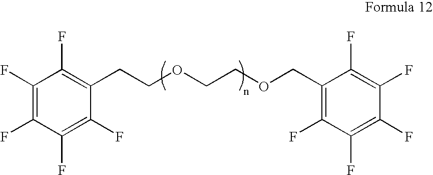
wherein n is from 3 to 20; 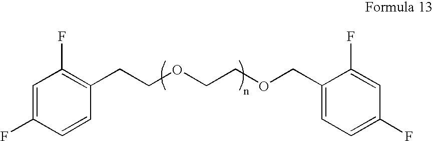
wherein n is from 3 to 20; 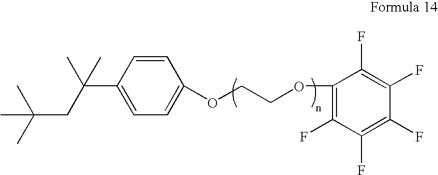
wherein n is from 3 to 20.

8. A carbon nanotube composition comprising the dispersant of claim 1, carbon nanotubes, and a solvent.

9. The carbon nanotube composition according to claim 8, wherein the carbon nanotubes are selected from single-walled carbon nanotubes, double-walled carbon nanotubes, multi-walled carbon nanotubes, rope carbon nanotubes, and combinations thereof.

10. The carbon nanotube composition according to claim 8, wherein the solvent is at least one kind selected from the group consisting of: water; alcohols, including methanol, ethanol, isopropyl alcohol, propyl alcohol and butanol; ketones, including acetone, methyl ethyl ketone, ethyl isobutyl ketone and methyl isobutyl ketone; glycols, including ethylene glycol, ethylene glycol methyl ether, ethylene glycol mono-n-propyl ether, propylene glycol, propylene glycol methyl ether, propylene glycol ethyl ether, propylene glycol butyl ether and propylene glycol propyl ether; amides, including dimethylformamide and dimethylacetamide; pyrrolidones, including N-methylpyrrolidone and N-ethylpyrrolidone; dimethylsulfoxide; γ-butyrolactone; hydroxyesters, including methyl lactate, ethyl lactate, methyl β-methoxyisobutyrate and methyl α-hydroxyisobutyrate; anilines, including aniline and N-methylaniline; hexane; terpineol; chloroform; toluene; propylene glycol monomethyl ether acetate (PGMEA); and N-methyl-2-pyrrolidone (NMP).

11. The carbon nanotube composition according to claim 8, wherein the composition comprises 0.001 to 10 parts by weight of the dispersant, 0.01 to 5 parts by weight of the carbon nanotubes and 85 to 99.989 parts by weight of the solvent, based on 100 parts by weight of the composition.
